(19) United States
(12) Patent Application Publication
(10) Pub. No.: US 2022/0125405 A1
(43) Pub. Date: Apr. 28, 2022

(54) System and Methods for Ultrasound Imaging with Modularized Frontend and Personal Computer System

(71) Applicant: Yanwei Wang, Bellevue, WA (US)

(72) Inventor: Yanwei Wang, Bellevue, WA (US)

(21) Appl. No.: 17/151,122

(22) Filed: Jan. 16, 2021

Related U.S. Application Data
(60) Provisional application No. 63/094,897, filed on Oct. 22, 2020.

(51) Int. Cl. A61B 8/00; A61B 8/14; A61B 8/08; G16H 30/40; G16H 40/63; G06F 13/42

(52) U.S. Cl. CPC A61B 8/4411; A61B 8/14; A61B 8/54; A61B 8/5207; G06F 2213/0026; G16H 40/63; G06F 13/4282; A61B 8/4427; A61B 8/4444; G16H 30/40

(57) ABSTRACT

A modularized ultrasound apparatus utilizes a PC system such as PC case, thermal management subsystem, power supply unit, motherboard, CPU, memory, hard drive, GPU, to build an ultrasound system by inserting frontend modules integrated on PCIe expansion cards as modularized components into the PC system's PCIe expansion subsystem.

### CROSS-REFERENCE TO RELATED APPLICATIONS

Provisional Application: Application No. 63/094,897

### REFERENCES CITED

1. “Ultrasound Open Platforms for Next-Generation Imaging Technique Development”, E Boni, C H Alfred, S Freear, J A Jensen, P Tortoli, IEEE Transactions on Ultrasonics, Ferroelectrics, and Frequency Control, Volume: 65, Issue: 7, July 2018, pp 1078-1092.
2. “ULA-OP: An Advanced Open Platform for Ultrasound Research”, Piero Tortoli, Luca Bassi, Enrico Boni, Alessandro Dallai, Francesco Guidi, Stefano Ricci, IEEE Transactions on Ultrasonics, Ferroelectrics, and Frequency Control, Volume: 56, Issue: 10, July 2018, pp 2207-2216.
3. J. A. Jensen et al., “SARUS: A synthetic aperture real-time ultrasound system,” IEEE Trans. Ultrason., Ferroelectr., Freq. Control, vol. 60, no. 9, pp. 1838-1852, September 2013.
4. C. C. P. Cheung et al., “Multi-channel pre-beamformed data acquisition system for research on advanced ultrasound imaging methods,” IEEE Trans. Ultrason., Ferroelectr., Freq. Control, vol. 59, no. 2, pp. 243-253, February 2012.
5. U.S. Pat. No. 5,795,297, “Ultrasonic Diagnostic Imaging System with Personal Computer Architecture”
6. Telemed, http://www.pcultrasound.com/
7. Cephasonics, https://www.cephasonics.com/
8. U.S. Pat. No. 8,824,743, “Adaptive ultrasound image reconstruction based on sensing of local media motion”
9. https://verasonics.com/wp-content/uploads/2019/04/Vantage-Systems-Brochure.pdf
10. M. Lewandowski, M. Walczak, B. Witek, P. Kulesza, K. Sielewicz, “Modular & Scalable Ultrasound Platform with GPU Processing,” 2012 IEEE International Ultrasonics Symposium, 7-10 Oct. 2012
11. http://us4us.eu/product/us4r/
12. https://on-demand.gputechconf.com/gtc/2018/presentation/s8421-ultrasound-medical-imaging-in-the-gpu-era.pdf

### FIELD OF THE INVENTION

The present invention relates to the field of ultrasound apparatus, systems, and methods for imaging and treatment. Specifically, the present invention relates to reducing cost, improving scalability, improving flexibility, improving computational power, of ultrasonic diagnostic or treatment apparatus.

### BACKGROUND OF THE INVENTION

An ultrasound imaging apparatus can be divided into two main subsystems. 1) A frontend subsystem responsible for: a) transmit signal generation and excitation of a probe; b) receiving signal, conditioning, and digital sampling; c) coordinating transmit and receive; d) send analog-to-digital convert (ADC) sampled data back to an imaging processor. A Frontend subsystem usually has a high number of physical channels, such as 128. It is common to build a 128 channel frontend system by combining a few, such as 4, PCBs together inside a customized box, where each PCB has 32 channels. The received data from these channels will be processed to construct the image. 2) Imaging processor subsystem is responsible for process the Frontend received data via beamforming, mid-end signal conditioning and processing, back-end image processing to generate the image ready for display. The most computationally intensive part of this imaging processor subsystem is beamforming. Historically for premium or highend system, beamforming is implemented by ASIC or FPGA. Diagrams of such architectures are available online. See for example, https://www.ti.com/solution/ultrasound-scanner, for various designs.

From development cost point-of-view, to develop an ultrasound imaging system with premium capability from scratch, a company or person faces the challenges of high development cost associated with building teams of 1) electronic hardware engineers to design and produce analog and digital circuits, 2) firmware engineers to drive these circuits and chips, 3) software engineers to work on various part of the system such as implementation of image reconstruction and processing algorithms, program control path, design UI, etc, 4) mechanical engineers to develop various modules of the system, such as system box/chassis and assemble the parts together.

For a company or person to develop a complete highend or premium capable ultrasound imaging system without high development cost illustrated above, there are two main approaches which are described in details below, as well as their short comings.

The first option is OEM ultrasound developed by some manufacture[6, 7], which is an ultrasound box integrated with frontend as well as hardware based beamforming. The ultrasound box will need to connect to a PC. The post processing could be located either in ultrasound box or in PC. This option works if the customer's purpose is to incorporating ultrasound image into his/her product, usually an application based on ultrasound image, without the need to develop his/her own ultrasound system. With this option, the customer cannot develop custom algorithms such as novel beamforming, since the hardware based beamforming has already been done inside the OEM box and pre-beamforming data is lost. For this case, since the post processing has already done in the OEM box or PC, there is not much can be done to develop your own ultrasound imaging system such advanced method/algorithm to improve image quality.

The second option is research ultrasound [1]-[4] developed in academia as well as industry[9][11]. These systems are for ultrasound research instead of routine diagnostic use. Research ultrasound[1]-[4][9] is an ultrasound box sitting outside of a computer and connected to the computer via high-speed links such as PCIe or USB3.0. The box either includes ultrasound front-end only which sends pre-beamformed data back to the computer [9][11], or together with beamforming module which sends beamformed data back to the computer, or with post-processing module as well which sends post-processed display ready data back to the computer. Research ultrasound provides the opportunity for a developer to design and implement his/her own specialized ultrasound system with software API to program the system.

Both the two options above have high hardware and development cost, no flexibilities in system size and shape, poor scalability of channel counts.

Besides ultrasound imaging apparatus, the current invention can also be applied to the following two cases. 1) In the case of an ultrasound treatment apparatus where the only purpose is deliver ultrasound energy into a tissue, the frontend subsystem, which drives a treatment transducer for delivering ultrasound energy, is responsible for only transmit signal generation and excitation of the probe without the implementation of receive related functionalities, such as those listed in the ultrasound imaging apparatus above includes: b) receiving signal, conditioning, and sampling; c) coordinating transmit and receive; d) send ADC sampled data back to an imaging processor. An ultrasound treatment apparatus does not have an imaging processor subsystem. 2) In the case of an ultrasound imaging as well as treatment apparatus, it includes both ultrasound imaging apparatus as well as ultrasound treatment apparatus.

### SUMMARY

The present inventor has realized the problems associated with the two approaches reviewed in the previous section for building an ultrasound system from scratch.

For the first option of OEM ultrasound developed by some manufacture[6, 7], two-box design make the system less compact and special hardware may be needed to secure and combine them together in case one combined system is desired. (The PC box can take the form factor of either a regular PC box, a laptop, or a small form factor PC). When other customized hardware is needed, it can be plugged into the PC's extension slot. This leads to a regular PC box needed (laptop and small form factor PC cannot have extension card added) which will be paired with the ultrasound box to build the complete system.

For the second option of research ultrasound [1]-[4][9][11], due to the lack of using off-the-shelf components and not optimized hardware design, such platform usually have high hardware (BOM) and manufacturing cost, bulky appearance, custom designed box and thermal management system, separated parts of data acquisition unit and data processing unit loosely connected to make the whole system. Research ultrasound [10]-[12] is using a single box design though, however, it still requires custom designed frontend PCB, custom box, and custom thermal management system, without the flexibility in choosing size and shape of the system box.

Both options above does not provide some or all of the following desired aspects for an ultrasound imaging system: 1) low hardware BOM cost, 2) low hardware development cost, 3) enough flexibilities of design of system size and shape, 4) enough scalability to build high channel count systems. Therefore, what is needed are systems and methods that overcome the above mentioned disadvantages.

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” or “another embodiment” or “some embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

The present disclosure details various aspects of a new paradigm for the design and building of a complete premium capable ultrasound imaging and/or treatment system. A modularized ultrasound frontend module is designed to have a PCIe add-on card form factor which can be inserted onto any PCIe slot of a PCIe expansion subsystem to finish the hardware build of an ultrasound system. The digital data generated from Frontend is sent to the CPU/GPU memory through PCIe or other high speed connections.

In one embodiment, the PC system's PCIe expansion subsystem is the expansions slots on the PC's motherboard. The PC system's PCIe expansion subsystem box is a PC case. As shown in FIG. 2.

In another embodiment, the PC system's PCIe expansion subsystem is the PCIe expansion slots of a plurality of PCIe expansion enclosures, where the PCIe expansion enclosures connect to said PC with high speed connections. The PC system's PCIe expansion subsystem box is the PCIe expansion enclosure case. As shown in FIG. 4. Each PCIe expansion enclosure will be a HCCM. Multiple HCCMs can be used to build high channel count system such as 1024 or 2048. The PCIe expansion enclosures are commercially available products.

In another embodiment, the PC system's PCIe expansion subsystem include a combination of both the PC's motherboard with PC case and PCIe expansion enclosures.

In some embodiment, one or multiple simplified PCIe Frontend modules, which has only transmit circuit present and transmit beamforming implemented on the control FPGA, can be inserted either inside a PC case or into one or multiple separate PCIe expansion enclosures to drive a treatment transducer and hence build an ultrasound treatment system.

In some embodiment, some of the Frontend modules are connected to an imaging probe, and at the same time, some of the Frontend modules are connected to an treatment probe, hence build a combined ultrasound imaging as well as treatment system.

With low cost frontend module and usage of mass produced off-the-shelf consumer electronic components such as GPU, PC chassis/case, motherboard, memory and hard drive, power supply, PCIe enclosure, etc, the disclosed ultrasound system can simultaneously achieve previously conflicting advantages: 1 low hardware cost, 2 low development cost, 3 great flexibility in choosing system form factor with existing off-the-shelf cases and PCIe enclosures, 4 scalability to extend to high channel count systems (higher than conventional 128 or 256 channel) by using existing PCIe enclosures, 5 high computational power and easily upgradable to latest technology.

### BRIEF DESCRIPTION OF THE DRAWINGS

The present invention is illustrated by way of example and not limitation in the figures of the accompanying drawings in which like references indicate similar elements.

FIG. 1 illustrates a Frontend PCIe module, according to one exemplary embodiment of the present invention.

FIG. 2 illustrates a top view diagram of a completely assembled system, according to one exemplary embodiment of the present invention.

FIG. 3 illustrates a top view diagram of an exemplary embodiment of a high channel count module (HCCM).

FIG. 4 illustrates another completed system with high physical channel count and high computation power, according to one exemplary embodiment of the present invention.

FIG. 5 illustrates PCB design of the frontend PCIe card, according to one exemplary embodiment of the present invention.

FIG. 6 illustrates FPGA frontend control system design block diagram, according to one exemplary embodiment of the present invention.

FIG. 7 illustrates a diagram of the master high channel count module (HCCM)'s clock and synchronization system. A HCCM subsystem configured with multiple frontend PCIe modules interconnected, according to one embodiment of the present invention.

FIG. 8 illustrates a diagram of the slave high channel count module (HCCM)'s clock and synchronization system, according to one embodiment of the present invention.

FIG. 9 illustrates an embodiment of the probe connector convertor, which connects two Frontend Modules and one ultrasound probe, according to one embodiment of the present invention.

### DETAILED DESCRIPTION OF THE INVENTION

Recent introduction of GPU into the ultrasound industry has the opportunity to significantly reduce the hardware and development cost to develop an ultrasound system. As the main processing unit, compared to the alternative technologies (previous approaches) using ASIC, FPGA, specialized DSP, and CPU, GPU can reduced the cost by magnitudes due to its low hardware cost per computational power as an off-the-shelve consumer product and extremely low development cost as only GPGPU programming is needed. For example, the ultrasound beamformer can be implemented by using off-the-shelve consumer grade GPU with magnitude of reduction in hardware cost compare with industry grade ASIC, FPGA, DSP, not to mention that the reduction of development cost is even more. It opens up extremely fast approaches to develop new ways to implement beamforming, image reconstruction, new imaging mode, other innovations, etc. It also provides a convenient low cost way to extend system's computational power by adding additional GPU cards, or simply upgrade to the latest GPU model.

The high data speed of PCIe extension bus makes the transfer of the RF pre-beamformed data into CPU or GPU memory become feasible. For example, a Frontend module with 64 channels with 64 MHz sample rate ADC and sample size of 16 bits will generate 8 GB data per second. This high data rate can be achieved by, for example, PCIe 3.0 x8.

Recent advances of the ultrasound frontend integrated circuits (IC) with highly integrated off-the-shelf frontend chips for transmit, receive, and analog to digital converter (ADC) make the development of compact ultrasound frontend module within a small form factor such as a PCIe card become feasible. The prevalence of PCIe IP controller on low end FPGA makes the low cost frontend card possible. Now it is practical to produce low cost, highly integrated frontend module in a form factor of a PC extension card.

The present inventor has 1) observed above evolutions; 2) realized that to build a premium capable ultrasound system, the only parts needs to be built is the Frontend: Image Processor and other peripheral subsystems can be assembled via off-the-shelve components; and 3) invented a modularized way to develop ultrasound system with low hardware and development cost, as well as high scalability of physical channels and computational power.

To achieve these improvements, an ultrasound frontend module is invented with a PCIe extension card form factor which can be inserted into a motherboard PCIe slots, sit inside and secured to the PC case without design and manufacture of custom system box and thermal management system. One or multiple frontend module can work together as a frontend subsystem to achieve various physical channel configurations. An ultrasound probe can be connected directly to the connector sitting on the frontend card's PCI bracket, or through a probe connector adaptor, especially when multiple frontend cards are involved.

With modularly designed system under the standard PC high speed expansion bus (PCIe) architect, current invention provides a fast and convenient way to develop ultrasound imaging systems with: flexibility of choosing system form factor, size, and shape; scalability to extend to higher channel counts; lowest cost by maximum using off-the-shelve consumer components; best computational power by free choice of the latest GPU model and number of GPUs.

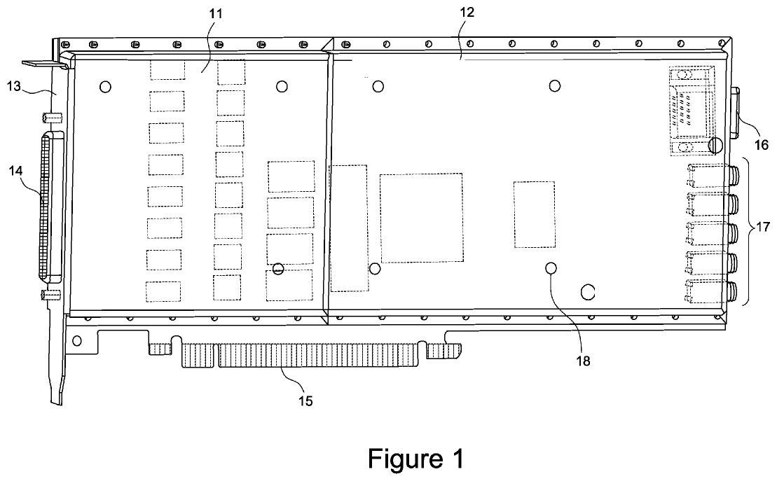

FIG. 1 illustrates diagram of an embodiment of a Frontend Card (Module): transmitter, receiver amplifier, A/D converter, FPGA controller, memory block, Clock circuitry, power regulator, all located on a PCIe card form factor, with a high element count connector 14 exposed outside of the PCIe bracket 13. The PCIe gold finger is shown as 15, Shield boxes for analog and digital circuits are shown as 11 and 12, respectively. The power connector 16 is used to connect to an outside power supply. A group of 5 coax connectors 17 are used for clock signal input/output, synchronous trigger signals input/output. Note that as shown in the figure, the PCI bracket and the shielding box are generated from the same piece of sheet of shielding material.

Using a standard or customized PC case as system box has following advantages. 1) As an off-the-shelf component, it has low hardware cost and zero development cost. 2) It is flexible to choose the size and shape of the system, as well as other components such as mother board, power supply, etc. Closed shielding enclosure is used for the frontend card to avoid environmental EM interferences inside of PC case. The cooling fans of the PC chassis/case's thermal system and the cooling fans/heat sink on the Frontend module work as the thermal management system to keep the frontend modules and the whole system from overheat. The ultrasound pre-beamformed data collected by the frontend will be transferred to CPU or GPU memory through PCIe bus. Power regulators are used to convert the 12V, 5V, and other power output from the power supply unit (PSU) into various analog and digital voltage rails used by the frontend module. These power regulators can be located either on each frontend module or on a separate Frontend Power Supply Module (FPSM) which also has a PCIe form factor and could be access and controlled by CPU.

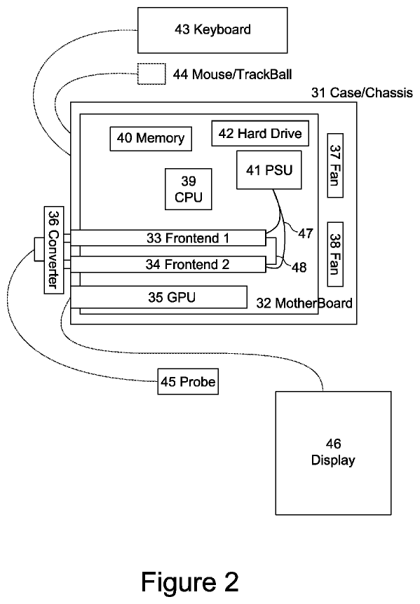

FIG. 2 illustrates top view diagram of an embodiment of a completely assembled system for implementing an apparatus of the present disclosure: a complete PC system with two Frontend modules plugged into its motherboard high-speed extension slots. All the standard parts for a PC system are included, such as a case 31, power supply unit (PSU) 41, cooling fans 37 and 38, hard drive 42, motherboard 32, GPU 35, CPU 39, Memory 40, display 46, keyboard 43, trackball 44. Only non existing off-the-shelve components are the two Frontend Modules 33, 34 and the probe connector 36. Probe 45 is connected to the probe connector.

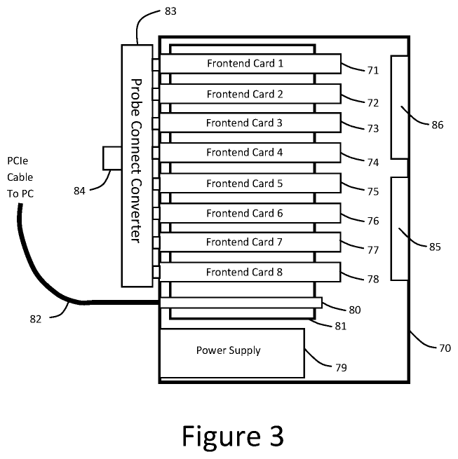

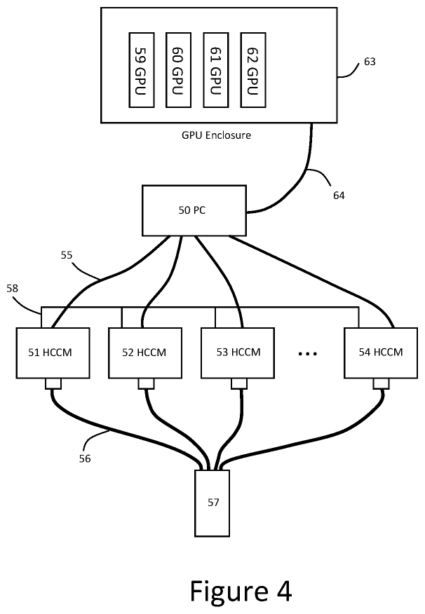

The idea of PC based ultrasound was first patented in [5], where a workstation or PC's CPU(s) is(are) used to perform the most of the image processor's work to get the image, instead of conventionally a series of serially connected special purpose circuits (made out of ASIC or FPGA), including beamforming, midend signal processing, and backend image processing, at the time of the invention around 1990s. One illustration of PC based ultrasound is shown in FIG. 3 of [5], which is a very high level illustration without implementation details such as whether the beamformer 90 in FIG. 3 is inside or outside of the PC case, or how to achieve it if the beamformer is inside the PC case. Another embodiment of [5] is shown in FIG. 4 of [5], where the complete system is overly-complex and still need a custom ultrasound system box to have everything included.

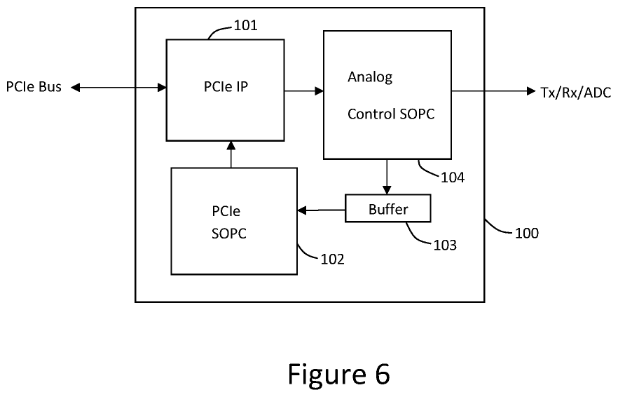

U.S. Pat. No. 8,824,743 [8] has mentioned an implementation of Frontend circuit as “plug-in module” which connect to the PC's expansion slot, as illustrated in FIG. 6 of [8] but it has several important differences with the proposed invention. The author of [8] is advocating the “separate ultrasound data collection box design” and teaches away from the current invention. See [9] for more information about the Vantage system. The “plug-in module” disclosed in [8] is the Vantage Data Acquisition system shown as the big box in the left next to the host computer in Figure “Verasonics Vantage Research Ultrasound System” of [9]. Below are the list of differences: 1) The “plug-in module” custom hardware is preferably housed in a custom enclosure, which is an ultrasound frontend box sitting outside of a computer and connect to the computer with high speed links. 2) A large block of expansion memory is used as part of the custom “plug-in module” hardware to store the RF data from the frontend circuit. Since this expansion memory on the “plug-in module” is accessed by the CPU during the “pixel-based reconstruction” process, it needs to store the RF data composed of an image frame. According to the calculation in [8], each transmit/receive event will use 1 MB data for a 128 channel system, and a typical image RF data from 256 events will need 256 MB memory to store. This large block of extra expansion memory is not needed in the proposed invention's frontend module, because the 256 transmit/receive event's data is send to PC system's memory or GPU's memory immediately after each transmit/receive event without accumulating all of them, hence 1 MB data memory is enough which corresponding to a single transmit/receive event. Compare to [8], the proposed invention's frontend modules need to store as low as 1 MB data corresponding to a single transmit/receive event, which is two magnitudes reduction from [8]'s 256 MB. For a typical system with 4 frontend modules to compose a 128 channel system, each frontend module only need 0.25 MB storage for the data. This can be easily accommodated by current lower end FPGA's internal logic or a small SRAM outside of FPGA. Hence reduces cost. 3) The space inside a PC box is a strong electromagnetic (EM) radiation environment. To protect the analog circuits in the frontend, shielding box is needed to void noise/artifacts in the collected data due to the EM interference. So shielding box on PCB is preferred to the design of the current invention where the frontend PCB needs to sit inside PC box. 4) [8]'s “plug-in module” requires an external enclosure to host multiple PCBs with each PCB is has a fixed number of physical channels. This requires design and build extra circuits inside the enclosure to aggregate the data from each PCB and send the aggregated data back to PC. However, the current invention will not require this aggregation circuit since each individual Frontend module will send its data to PC or GPU memory directly and independently. Hence significantly simplifies the architecture and reduces cost. 5) [8] doesn't include the clock and transmit/receive event synchronization signal on each frontend module with off-the-shelve connectors which enables the building of a system with scalable number of Frontend modules without the need of building a customized external enclosure since in [8] the external synchronization signal is on the external enclosure. Note the “plug-in module” in [8] is different from the frontend module in the current invention with above 5 differences. The current invention provides reduced system cost, reduced development cost, and better flexibility in building customized systems compare to [8]. Note that in another embodiment of the current invention, where very high channel count requires multiple boxes to hold a large number of frontend modules, the frontend module design enables the use of existing off-the-shelve PCIe enclosure to hold multiple frontend modules and send the aggregated data back to PC system, this approach still has the above differences 2)˜4) compared with [8]. Although this embodiment also using external box, these box are off-the-shelve PCIe enclosure and hence reduced development cost associated with the customized enclosure in [8].

To meet the requirements of medical device regulation, medical degree off-the-shelve power supply unit can be used for this purpose and avoid delay to the market.

The invented platform also enables a new market: individuals, who do not have the resources to develop a complete ultrasound system, can develop their own state-of-the-art ultrasound system by plugging in frontend cards into a PC system and connect an ultrasound probe. The standard accessories and modularized hardware and software enables developers to develop or customize their own systems by off-the-shelve components. There is significant flexibility of the system developed by using the disclosed platform. For example, when multiple frontend cards are used, the clock and trigger signals from the master needs to be connected to other slaves, which can be solved by using off-the-shelve Y cables. With PCIe card form factor based frontend module as off-the-shelf component, developer of ultrasound system can simply choose a few frontend cards, two for example, plug them into the existing PC case, as well as GPU cards for high computation power and good system performance, to finish up the hardware development of the system.

Another embodiment of the current invention is building high channel count system by using off-the-shelve PCIe enclosures. One PCIe enclosure can be used to hold multiple frontend modules, hence is called a high channel count module (HCCM). One PCIe enclosure can also be used to hold multiple GPUs. To scale the system in both physical channels and computational power, the system just need to increase the number of HCCM and GPU enclosures.

FIG. 3 illustrates a top view diagram of an embodiment of a high channel count module (HCCM) which is implemented by a PCIe extension enclosure 70. In the diagram, there are 8 PCIe Frontend modules are used, 71 to 78. All these Frontend modules are plugged in the PCIe slots on the extension board 81 inside the enclosure 70. An upstream adapter 80 is commonly used to connect the PCIe enclosure system to a Host via PCIe extension cable 82. Such enclosures has a power supply unit 79. A probe connect adaptor/converter 83 is configured to connect to all the Frontend modules at one side, and at the same side, a probe connector 84 to connect to one or multiple probes.

FIG. 4 illustrates a diagram of an embodiment of a complete high channel count system with one or multiple high channel count modules (HCCM) 51˜54: all the extension enclosures are connected to the PC with high-speed bus 55 such as PCIe, Thunderbolt, or other high speed connection. Probe connection cables 56 are used to connect probe 57 to each HCCM. A clock and sync signal coax network 58 is used to interconnect all the HCCMs. For example, if each Frontend module has 64 physical channels, each HCCMs will have 256 physical channels, and the 4 HCCMs system in FIG. 4 will have 1024 physical channels. Clearly, a 2048 physical channel system can be build by using 8 HCCMs, etc. A high power processor module (HPPM) 63 is illustrated in the system: a PCIe enclosure 63 is used to host 4 GPUs 59˜62. The HPPM is connected to the PC with high speed connection cable 64. Note that the figure is only for one embodiment of high physical channel count and how power processor system. The locations of each individual Frontend module and GPU are not fixed, they can be mixed located in any PC or PCIe enclosures.

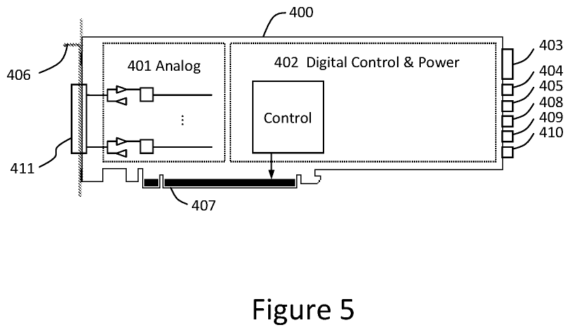

FIG. 5 illustrates the PCB layout of an embodiment of the Frontend card. The PCB 400 is in a PCIe form factor, where the analog circuit 401 such as transmit, receive, high voltage switch, and ADCs are located close to the PCIe bracket 406, which is also shown in FIG. 1 as item 13. The digital circuit 402 are located at the opposite side of the PCIe bracket 406. Power connector 403, clock input/output connector 404, and synchronization signal input/output connector 405,408,409, and 410 are shown. 407 is the PCIe gold finger. 411 is the connector on frontend module to connect to probe.

FIG. 6 illustrates a block diagram of an embodiment of the frontend FPGA control system 100 in the Frontend module. A PCIe IP 101 is used to connect to the system PCIe bus to receive and send data between Frontend module and CPU or GPU. An analog control system-on-a-programmable-chip (SOPC) 104, also known as soft CPU, can receive configurations of an imaging mode from CPU through PCIe IP, and generate each transmit, receive, ADC, and save the data into a buffer 103. An PCIe SOPC 102 is configured to access the common buffer 103, generate DMA sequence and drive the PCIe IP 101 in the FPGA to send the DMA data back to CPU/GPU memory through system PCIe bus. The analogy control 104 and the PCIe control 102 are designed to use separate clock domains. As a bridge, the common buffer 103 can be accessed by both Analog control SOPC 104 and PCIe control 102, in a form of dual port memory.

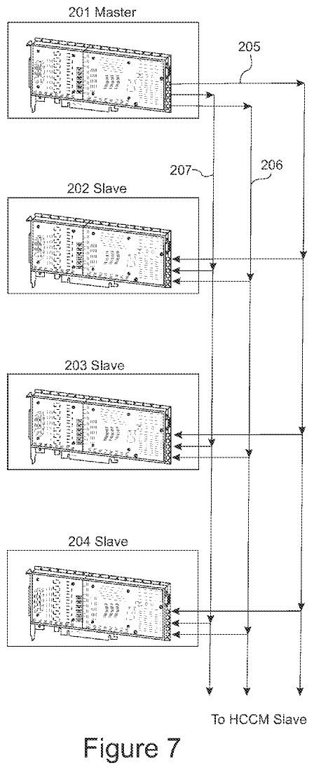

FIG. 7 illustrates a diagram of an embodiment of the Master HCCM's synchronization system. One Frontend Module 201 is either hardware or software configured as Master and the other three Frontend Model 202˜204 are configured as slaves. The master card will output clock signal 205 which is sent to other slave modules to use. Master card will also generate frame trigger 206 signals which trigger slave cards to start a new frame. Master card will also generate a line trigger 207 which trigger slave cards to start a new transmit and receive event. All the Frontend cards will send their received data individually to the CPU/GPU memory. The off-the-shelve coax cable or Y cable could be used for these interconnection between Master and Slaves. This is only an explanatory embodiment of clock and synchronization signals design.

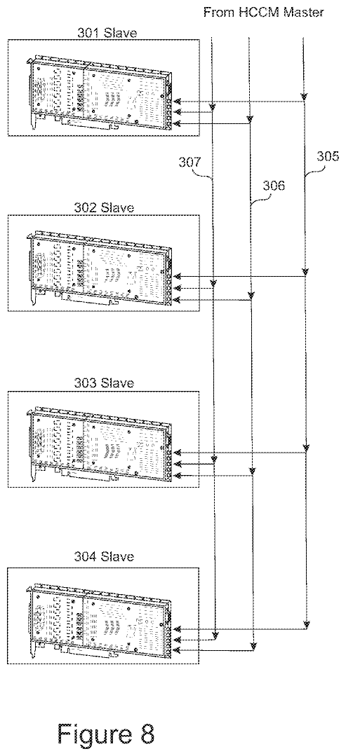

FIG. 8 illustrates a diagram of an embodiment of the Slave HCCM's synchronization system. All the Frontend Modules 301˜304 are slave. The clock 305 and trigger signals 306,307 are all from a Master HCCM.

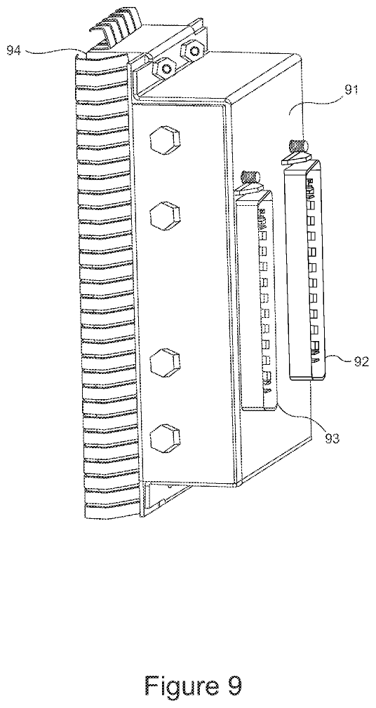

FIG. 9 illustrates a diagram of an embodiment of the probe adapter (probe connector convertor) 91. two high element count connectors 92 and 93 are used to connect to two Frontend modules. A ultrasound probe connector 94 are located on the other side of the probe connector converter. Proper shielding materials are used to enclose these connectors.

It will be apparent to those skilled in the art that various modifications and variations can be made to the disclosed embodiments. It is intended that the specification and examples be considered as exemplary only, with a true scope of the disclosure being indicated by the following claims and their equivalents.

1. An ultrasound imaging apparatus comprising:
a frontend subsystem configured to control the operation of a probe connected to said frontend subsystem by transmitting and receiving ultrasound wave into the target of interest, condition and digitize the received signal, and send the digital data into an imaging processor; and
a PC system configured to implement the image processor to generate at least one ultrasound image;
wherein the frontend subsystem is further configured to have a single or a plurality of frontend module(s) of the form of computer high-speed bus PCIe card(s) which is(are) plugged into a single or a plurality of high-speed PCIe expansion slot(s) of the PC system's PCIe expansion subsystem.
wherein said frontend module's PCIe form factor matches PCI slot of said PC system's PCIe expansion subsystem box and secures itself to said PC system's PCIe expansion subsystem box when plugged into the PCIe expansion slot of said PC system.

2. The ultrasound imaging apparatus of claim 1, wherein the frontend module is configured to has a connector sit on the PCIe bracket,
wherein said connector is used to connect to a probe outside of the PC case.

3. The ultrasound imaging apparatus of claim 1, wherein the frontend module is configured to have power input, clock and synchronization signal input or output.

4. The ultrasound imaging apparatus of claim 1, wherein the frontend module is configured to have a FPGA to control the transmit and receive of the ultrasound signals as well as sending the ADC data to the CPU or GPU through PCIe connector.

5. The ultrasound imaging apparatus of claim 4, wherein the frontend module is further configured to send per transmit/receive event ADC data to CPU or GPU memory.

6. The ultrasound imaging apparatus of claim 4, wherein the FPGA is a low end FPGA control unit with PCIe IP controller.

7. The ultrasound imaging apparatus of claim 4, wherein the FPGA is further configured to have a Soft CPU implemented on the FPGA to implement: a) transmit signal generation and excitation of a probe; b) receiving signal, conditioning, and ADC sampling; c) coordinating transmit and receive;
wherein the FPGA is further configured to have a soft CPU implemented on the FPGA to send ADC sampled data back to the imaging processor.

8. The ultrasound imaging apparatus of claim 7, wherein a dual port data buffer is used to work as a common buffer bridge between these two soft CPUs.

9. The ultrasound imaging apparatus of claim 1, wherein the frontend module is further configured to have an EM shielding structure to protect the sensitive circuit from EM interferences inside PC system's PCIe expansion subsystem box

10. The ultrasound imaging apparatus of claim 9, wherein the EM shielding structure and the PCI bracket are made out of one piece of metal.

11. The ultrasound imaging apparatus of claim 1, wherein the frontend module can be configured as either master or slave;
wherein the master frontend module is configured to output clock signal and synchronization signal;
wherein the slave frontend module can receive clock signal and synchronization signal.

12. The ultrasound imaging apparatus claim 1, further comprising: a probe adapter module with shielding box wherein the probe adapter connect to the frontend subsystem through the connector on the PCIe bracket of the frontend module;
wherein the probe adapter has a probe connector interface which is used to connect to a probe.

13. The ultrasound imaging apparatus claim 1, further comprising: a probe adapter subsystem which has multiple cables and connectors pairs where some cable/connector pair connects to the connector on the frontend module, some cable/connector pair connects to each individual probe, switches are used to selectively connect different probes to the frontend modules.

14. The ultrasound imaging apparatus of claim 1, wherein the image processor subsystem is further configured to have Pre-Allocate buffers in host memory for receiving data from frontend modules during the startup of the PC.

15. The ultrasound imaging apparatus of claim 1, wherein the PC system is configured to have a PSU module;
wherein the PSU can use medical grade PSU to meet medical regulations.

16. An ultrasound treatment apparatus comprising: a treatment frontend subsystem configured to control the operation of a probe connected to said frontend subsystem by transmitting ultrasound wave into the target of interest; and
a PC system configured to control the frontend subsystem;
wherein the frontend subsystem is further configured to have a single or a plurality of Frontend module(s) of the form of computer high-speed bus PCIe card(s) which is(are) plugged into a single or a plurality of high-speed PCIe expansion slot(s) of the PC system's PCIe expansion subsystem.
wherein said Frontend module's PCIe form factor matches PCI slot of said PC system's PCIe expansion subsystem box and secures itself to said PC system's PCIe expansion subsystem box when plugged into the PCIe expansion slot of said PC system.

17. An ultrasound treatment as well as imaging apparatus comprising: an imaging frontend subsystem configured to control the operation of a probe connected to said frontend subsystem by transmitting and receiving ultrasound wave into the target of interest, condition and digitize the received signal, and send the digital data into an imaging processor;
a treatment frontend subsystem configured to control the operation of a probe connected to said frontend subsystem by transmitting ultrasound wave into the target of interest; and
a PC system configured to implement the image processor to generate at least one ultrasound image;
wherein the frontend subsystem is further configured to have a single or a plurality of Frontend module(s) of the form of computer high-speed bus PCIe card(s) which is(are) plugged into a single or a plurality of high-speed PCIe expansion slot(s) of the PC system's PCIe expansion subsystem.
wherein said Frontend module's PCIe form factor matches PCI slot of said PC system's PCIe expansion subsystem box and secures itself to said PC system's PCIe expansion subsystem box when plugged into the PCIe expansion slot of said PC system.
